(19) United States
(12) Patent Application Publication
(10) Pub. No.: US 2002/0155088 A1
(43) Pub. Date: Oct. 24, 2002

(54) Tumor treatment by using oligoaniline

(76) Inventor: Long Y. Chiang, Taipei (TW)

Correspondence Address: Y. ROCKY TSAO, Fish & Richardson P.C., 225 Franklin Street, Boston, MA, 02110-2804, US

(21) Appl. No.: 09/840,322

(22) Filed: Apr. 23, 2001

(51) Int. Cl. A61K 31/785; A61K 31/70

(52) U.S. Cl. 424/78.17; 514/42; 514/137; 514/517; 514/564; 514/568

(57) ABSTRACT

A method of inhibiting the growth of tumor cells in a tumor site of a subject. The method includes administering to the tumor site an oligoaniline and subsequently exposing the tumor site to irradiation.

### BACKGROUND OF THE INVENTION

Free radicals have been shown to inhibit tumor growth by causing oxidative damage to lipids, proteins, and nucleic acids of tumor cells. In clinical practice, a photo-sensitizer is first delivered to a tumor site and then activated by irradiation to generate free radicals, thus inhibiting tumor growth. Among known photo-sensitizers, Photofrin II has recently been approved by the U.S. Food and Drug Administration. Preparation of Photofrin II is tedious.

Oligoanilines, electron-rich molecules, are built up by the repetition of aniline units. When photo-excited, they are capable of generating free radicals which in turn convert molecular oxygen into singlet oxygen and related free radicals, such as O₂⁻. Oligoanilines have low bioavailability and must be chemically modified before they can be tested for their efficacy, if any, as photo-sensitizers in treating tumor.

### SUMMARY OF THE INVENTION

This invention relates to a method of inhibiting growth, including causing death, of tumor cells. The method includes administering to a tumor site of a subject in need thereof a photo-sensitive free radical-generating oligoaniline and, subsequently exposing the tumor site to irradiation. The oligoaniline, administered to the subject in an amount sufficient to inhibit the growth of the tumor cells at the tumor site, is of the following formula:

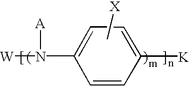

In this formula, m is an integer of 1-6; n is an integer of 1-10; each A is —H, —Z, —CH₂—CO—OH, —CH₂—CO—O—Z, —CH₂—CO—S—Z, —CH₂—CO—NH₂, or —CH₂—CO—NH—Z; and each X is —H, —O—Z, —S—Z, —NH—Z; Z being —E—D, wherein E is —R—, —R—Ar—, —Ar—R—, or —A—-; and D is —OH, —SH, —NH₂, —NHOH, —SO₃H, —OSO₃H, —CO₂H, —CONH₂, —CH(NH₂)—CO₂H, —P(OH)₃, —PO(OH)₂, —O—PO(OH)₂, —O—PO(OH)—O—PO(OH)₂, —O—PO(O⁻)—O—CH₂CH₂NH₃⁺, -glycoside, —OCH₃, —OCH₂(CHOH)₄—CH₂OH, —OCH₂(CHOH)₂—CH₂OH, —C₆H₃(OH)₂, —NH₃⁺, —N⁺H₂R_{b}, —N⁺HR_{b}R_{c}, or —N⁺R_{b}R_{c}R_{d}, each of R, R_{b}, R_{c}, and R_{d}, independently, being C₁₋₃₀ alkyl; and Ar being aryl; W is —H, —CO—B, —CH₂CH(OH)—B, —CO—NH—B, —CS—NH—B, —CO—O—B, CO—CH₂—CH(CO₂H)—B, —CH₂—B, —SO₂—B, wherein B is —R₁—O—[Si(CH₃)₂—O—]₁₋₁₀₀, C₁₋₂₀₀₀ alkyl, C₆₋₄₀ aryl, C₇₋₆₀ alkylaryl, C₇₋₆₀ arylalkyl, (C₁₃₀ alkyl ether)₁₋₁₀₀, (C₆₋₄₀ aryl ether)₁₋₁₀₀, (C₇₋₆₀ alkylaryl ether)₁₋₁₀₀, (C₇₋₆₀ arylalkyl ether)₁₋₁₀₀, (C₁₋₃₀ alkyl thioether)₁₋₁₀₀, (C₆₋₄₀ aryl thioether)₁₋₁₀₀, (C₇₋₆₀ alkylaryl thioether)₁₋₁₀₀, (C₇₋₆₀ arylalkyl thioether)₁₋₁₀₀, (C₂₋₅₀ alkyl ester)₁₋₁₀₀, (C₇₋₆₀ aryl ester)₁₋₁₀₀, (C₈₋₇₀ alkylaryl ester)₁₋₁₀₀, (C₈₋₇₀ arylalkyl ester)₁₋₁₀₀, —R₁—CO—O—(C₁₋₃₀ alkyl ether)₁₋₁₀₀, —R₁—CO—O—(C₆₋₄₀ aryl ether)₁₋₁₀₀, —R₁—CO—O—(C₇₋₆₀ alkylaryl ether)₁₋₁₀₀, —R₁—CO—O—(C₇₋₆₀ arylalkyl ether)₁₋₁₀₀, (C₄₋₅₀ alkyl urethane)₁₋₁₀₀, (C₁₄₋₆₀ aryl urethane)₁₋₁₀₀, (C₁₀₋₈₀ alkylaryl urethane)₁₋₁₀₀, (C₁₀₋₈₀ arylalkyl urethane)₁₋₁₀₀, (C₅₋₅₀ alkyl urea)₁₋₁₀₀, (C₁₄₋₆₀ aryl urea)₁₋₁₀₀, (C₁₀₋₈₀ alkylaryl urea)₁₋₁₀₀, (C₁₀₋₈₀ arylalkyl urea)₁₋₁₀₀, (C₂₋₅₀ alkyl amide)₁₋₁₀₀, (C₇₋₆₀ aryl amide)₁₋₁₀₀, (C₈₋₇₀ alkylaryl amide)₁₋₁₀₀, (C₈₋₇₀ arylalkyl amide)₁₋₁₀₀, (C₃₋₃₀ alkyl anhydride)₁₋₁₀₀, (C₈₋₅₀ aryl anhydride)₁₋₁₀₀, (C₉₋₆₀ alkylaryl anhydride)₁₋₁₀₀, (C₉₋₆₀ arylalkyl anhydride)₁₋₁₀₀, (C₂₋₃₀ alkyl carbonate)₁₋₁₀₀, (C₇₋₅₀ aryl carbonate)₁₋₁₀₀, (C₈₋₆₀ alkylaryl carbonate)₁₋₁₀₀, (C₈₋₆₀ arylalkyl carbonate)₁₋₁₀₀, —R₁—O—CO—NH—(R₂ or Ar—R₂—Ar)—NH—CO—O—(C₁₋₃₀ alkyl ether, C₆₋₄₀ aryl ether, C₇₋₆₀ alkylaryl ether, or C₇₋₆₀ arylalkyl ether)₁₋₁₀₀, —R₁—O—CO—NH—(R₂ or Ar—R₂—Ar)—NH—CO—O—(C₂₋₅₀ alkyl ester, C₇₋₆₀ aryl ester, C₈₋₇₀ alkylaryl ester, or C₈₋₇₀ arylalkyl ester)₁₋₁₀₀, —R₁—O—CO—NH—(R₂ or Ar—R₂—Ar)—NH—CO—O—(C₁₋₃₀ alkyl ether, C₆₋₄₀ aryl ether, C₇₋₆₀ alkylaryl ether, or C₇₋₆₀ arylalkyl ether)₁₋₁₀₀—CO—NH—(R₂ or Ar—R₂—Ar)—NH—CO—O—, —R₁—O—CO—NH—(R₂ or Ar—R₂—Ar)—NH—CO—O—(C₂₋₅₀ alkyl ester, C₇₋₆₀ aryl ester, C₈₋₇₀ alkylaryl ester, or C₈₋₇₀ arylalkyl ester)₁₋₁₀₀—R₃—O—CO—NH—(R₂ or Ar—R₂—Ar)—NH—CO—O—, —R₁—NH—CO—NH—(R₂ or Ar—R₂—Ar)—NH—CO—O—(C₁₋₃₀ alkyl ether, C₆₋₄₀ aryl ether, C₇₋₆₀ alkylaryl ether, or C₇₋₆₀ arylalkyl ether)₁₋₁₀₀, —R₁—NH—CO—NH—(R₂ or Ar—R₂—Ar)—NH—CO—(C₂₋₅₀ alkyl ester, C₇₋₆₀ aryl ester, C₈₋₇₀ alkylaryl ester, or C₈₋₇₀ arylalkyl ester)₁₋₁₀₀, —RI—NH—CO—NH—(R₂ or Ar—R₂—Ar)—NH—CO—O—(C₁₋₃₀ alkyl ether, C₆₋₄₀ aryl ether, C₇₋₆₀ alkylaryl ether, or C₇₋₆₀ arylalkyl ether)₁₋₁₀₀—CO—NH—(R₂ or Ar—R₂—Ar)—NH—CO—O—, —R₁—NH—CO—NH—(R₂ or Ar—R₂—Ar)—NH—CO—O—(C₂₋₅₀ alkyl ester, C₇₋₆₀ aryl ester, C₈₋₇₀ alkylaryl ester, or C₈₋₇₀ arylalkyl ester)₁₋₁₀₀—R₃O—CO—NH—(R₂ or Ar—R₂—Ar)—NH—CO—O—, —R₁—O—CO—NH—(R₂ or Ar—R₂—Ar)—NH—CO—NH—(C₂₋₅₀ alkyl amide, C₇₋₆₀ aryl amide, C₈₋₇₀ alkylaryl amide, or C₈₋₇₀ arylalkyl amide)₁₋₁₀₀, or —R₁—NH—CO—NH—(R₂ or Ar—R₂—Ar)—NH—CO—NH—(C₂₋₅₀ alkyl amide, C₇₋₆₀ aryl amide, C₈₋₇₀ alkylaryl amide, or C₈₋₇₀ arylalkyl amide)₁₋₁₀₀; wherein each of R₁, R₂, and R₃, independently, is C₁₋₃₀ alkyl; and Ar is aryl; K is —H, —[N(X)-C₆H₄]₁₋₃—NH₂, —[N(X)—C₆H₄]₁₋₃—NH—C(═S)—SH, —[N(X)—C₆H₄]₁₋₃—N═CH—Ar—SH, or —[N(X)—C₆H₄]₁₋₃—NH—CO—Ar—SH, wherein X is —H, —Z, —CH₂—CO—OH, —CH₂—CO—O—Z, —CH₂—CO—S—Z, —CH₂—CO—NH₂ or —CH₂—CO—NH—Z; and Ar is aryl.

The oligoanilines described above include their pharmaceutically acceptable salts, if applicable. Such a salt can be formed between a negatively charged ionic group in an oligoaniline (e.g., sulfonate or carbonate) and a positively charged counterion (e.g., sodium, potassium, calcium, or magnesium). Likewise, a positively charged ionic group in an oligoaniline (e.g., ammonium) can also form a salt with a negatively charged counterion (e.g., chloride, bromide, or iodide). Examples of such oligoaniline salts include sodium sulfobutylated tetraaniline, and aminomethylcarbonyl hexadecaaniline chloride.

An oligoaniline to be used to practice the method of this invention is formulated into a pharmaceutical composition prior to its use in tumor treatment. Thus, also within the scope of the invention is a pharmaceutical composition which contains such an oligoaniline and a pharmaceutically acceptable carrier for use in treating tumor. Examples of the carriers include water, colloidal silica oxide, magnesium sterate, lipid, lipoprotein, blood protein, or cellulose.

One of the above-described oligoanilines, as an active ingredient in a pharmaceutical composition, is first administered to a tumor site of a subject before the tumor site is exposed to irradiation. Upon irradiation, the oligoaniline converts surrounding molecular oxygen to highly reactive oxygen radicals, including superoxide radicals, which in turn attack the tumor cells and inhibit their growth.

Thus, the invention also relates to using one of the above-described oligoanilines for manufacture of a medicament for tumor treatment.

Details of the invention are set forth in the description below. Other features, objects, and advantages of the invention will be apparent from the description and from the claims.

### DETAILED DESCRIPTION OF THE INVENTION

This invention relates to use of an oligoaniline as a photodynamic therapeutic agent to inhibit growth of benign or malignant tumor cells. The oligoaniline is optionally substituted, either at one or more nitrogen atoms or at one or more benzene rings.

Oligoanilines to be used to practice the method of this invention can be synthesized by methods well know in the art, e.g., consecutive oxidative condensation of N-phenyl-1,4-phenylenediamine using ammonium peroxydisulfate [(NH₄)₂S₂O₈] as an oxidant. See Zhang et al. Synth. Met. 1997, 84, 119; and Wei et al. Synth. Met. 1997, 84, 289. Functionalized oligoanilnes can be synthesized either by direct polymerization of aniline derivatives initiated by peroxydisulfate dianion or by reductive nucleophilic addition on quinonoid moieties of oligoanilines with alkylamino or thiol nucleophiles. See U.S. Pat. No. 4,940,517 to Wei; and Han et al. Chem. Mater. 1999, 11, 480.

Oligoanilines are water-insoluble. The most commonly used approach for enhancing the solubility of oligoanilines is to convert them into their sulfonic acid derivatives by attaching sulfonyl groups on benzene carbon atoms benzene moieties or sulfoalkyl groups on the anilinic nitrogen atoms. See Hany et al. Synth. Met. 1989, 31, 369; Yue et al. J. Am. Chem. Soc. 1990, 112, 2800; Bergeron et al. J. Chem. Soc., Chem. Comm. 1990, 180; Yue et al. J. Am. Chem. Soc. 1991, 113, 2665; Nguyen et al. Macromolecules 1994, 27, 3625; and Nguyen et al. TRIP 1995, 3, 186.

A suitable oligoaniline or its salt in an effective amount is formulated with a pharmaceutically acceptable carrier to form a pharmaceutical composition before it is administered to a subject in need of tumor treatment. “An effective amount” refers to the amount of the compound which is required to confer therapeutic effect on the treated subject. The interrelationship of dosages for animals and humans (based on milligrams per square meter of body surface) is described by Freireich et al., Cancer Chemother. Rep., 1966, 50, 219. Body surface area may be approximately determined from height and weight of the patient. See, e.g., Scientific Tables, Geigy Pharmaceuticals, Ardley, N.Y., 1970, 537. Effective doses will also vary, as recognized by those skilled in the art, depending on the route of administration, the excipient usage, the distance of tumor from the skin surface, the source of the irradiation, and the optional co-usage with other therapeutic treatments including use of other anti-tumor compounds. Examples of pharmaceutically acceptable carriers include colloidal silicon dioxide, magnesium stearate, cellulose, sodium lauryl sulfate, and D&C Yellow #10.

The pharmaceutical composition may be administered via a parenteral route, e.g., topically, intraperitoneally, and intravenously. Examples of parenteral dosage forms include an active compound dissolved in phosphate buffer solution (PBS), or admixed with any other pharmaceutically acceptable carrier. Solubilizing agents, such as cyclodextrins, or other solubilizing agents well known to those familiar with the art, can also be included in the pharmaceutical composition.

An in vitro inhibition assay can be performed to preliminarily evaluate an oligoaniline's ability to inhibit the growth of tumor cells. For example, an oligoaniline solution is added to a pre-incubated cell suspension. Subsequently, the cell suspension is irradiated with fluorescence light, followed by further incubation. A 3-(4,5-dimethylthiazol-2-yl)-2,5-diphenyl-tetrazolium bromide solution is then added to the cell suspension to react with mitochrondrial dehydrogenase to form formazon, which is extracted with dimethyl sulfoxide (DMSO). The DMSO extract solution is immediately used for optical measurement to determine the formazon quantity, which correlates with the quantity of dehydrogenase or the relative number of the living cells.

Oligoanilines that show efficacy in preliminary studies can be further tested to confirm their efficacy by an in vivo assay on tumor-bearing mice. For example, see Chiang, et al. Proc. Electrochem. Soc., 1999, 99-12, 238-249. More specifically, a tumor-bearing mouse can be first administered with an oligoaniline to be tested in PBS close to the tumor site. The mouse is then kept in the dark while the oligoaniline is circulated to the tumor site. The tumor site is exposed by removing the hair on and around it, and then irradiated with a laser beam or other light source. After the irradiation, the growth of the tumor in the mouse is examined at different time intervals. The inhibitory effect can be evaluated by measuring the body weights of the mouse and the volumes of the tumor. After the mouse is sacrificed, the final body weight, the weights of various organs, and the final volume and weight of the tumor are measured; and blood is collected for biochemistry and hematology analyses. The data are evaluated used to determine the photodynamic therapeutic and other effects.

When photo-exicited, oligoanilines convert molecular oxygen into singlet oxygen and related free radicals, such as superoxide free radicals and hydroxyl radicals. The free radicals subsequently cause damage to surrounding tumor cells and inhibit the growth of the tumor cells (i.e., reducing the number and size of the tumor cells). The irradiation source can be laser or other lights, e.g., fluorescence or X-rays. The irradiation can be of a wavelength of 400-1000 nm and an energy intensity of 10-300 J/cm², and the irradiation time can be 10-200 minutes.

Without further elaboration, it is believed that one skilled in the art, based on the description herein, can utilize the present invention to its fullest extent. All publications recited herein are hereby incorporated by reference in their entirety. The following specific examples, which describe synthesis and biological testing of sulfobutylated hexadecaaniline which can be used in the present invention, are therefore to be construed as merely illustrative, and not limitative of the remainder of the disclosure in any way whatsoever.

### EXAMPLE 1

Synthesis of Water-Soluble Sulfobutylated Hexadecaaniline (A₁₆S)

500 mg hexadecaaniline and 50 mL dimethylformamide (DMF) were mixed in a dry flask equipped with a stirring bar and a condenser under N₂. To the DMF solution was then added 150 mg sodium hydride at −30° C. The mixture thus obtained was stirred for 30 minutes, mixed with 1.5 mL 1,4-butane sultone, allowed to slowly warm up to 70° C., and stirred for 6.0 hours. At the end of the stirring, 200 mL acetone was added to the reaction mixture to effect precipitation of a solid. The solid, which included A₁₆S and impurities, was collected, washed twice with acetone, and dissolved in H₂O. The aqueous solution was filtered through celite, and to the filtrate thus obtained was slowly added acetone again to effect precipitation of a solid. The solid was collected after centrifuge, washed with acetone, and dried under vacuum at 40° C. to yield 575 mg water-soluble A₁₆S.

IR γₘₐₓ (KBr): 3433 (br), 2953 (C—H), 2866, 1598 (amide, s), 1509, 1302, 1183 (s), 1046, 833, 742, 610, and 531 cm⁻¹.

¹H NMR δ (ppm, in D₂O): 1.67 (m, 4H), 2.90 (t, 2H), 3.56 (t, 2H), and 6.0-7.2 (aromatic).

### EXAMPLE 2

In vitro Irradiation-Induced Superoxide Generation by A₁₆S

The ability of A₁₆S to generate superoxide free radicals was demonstrated as follows: 1.0 mL 25 μM A₁₆S aqueous solution was added to 1.0 mL ferricytochrome C-containing PBS (50 μM). The mixture thus obtained was added into wells of a 24-well plate, and exposed to fluorescence light source (27 watts) for 0-90 minutes. The distance between the plate cover and the light source was 5-6 cm. The reduction extent of ferricytochrome C was evaluated based on its absorbance at 550 nm, which corresponded to the increase of the quantity of reduced ferrocytochrome C. More specifically, production of ferrocytochrome C indicated that A₁₆S, upon irradiation, converted molecular oxygen to superoxide free radicals, and electron transfer from the superoxide free radicals to ferricytochrome C reduced the ferricytochrome C to ferrocytochrome C. It was observed that, at a constant A₁₆S concentration, the amount of superoxide free radicals increased as the irradiation time increased from 0, 30, 60, to 90 minutes.

### EXAMPLE 3

In vitro Irradiation-Induced Cytotoxicity of A₁₆S Based on Tumor Cell Viability

Two types of tumor cells used in this study were prepared as follows: Fibrosarcoma cells (CCRC₆₀₀₃₇) and sarcoma 180 cells (obtained form Biochemical Institute of Chung Shan Medical and Dental College, Taiwan) were maintained and cultured in an cc-modified eagle medium (MEM) containing L-glutamine and phenol red, 10% fetal bovine serum, and antibiotics (100 units/mL of penicillin G and 100 μg/mL streptomycin sulfate). The cells were incubated in the dark in 95% humidified air plus 5% CO₂, harvested by treatment with trypsin-EDTA, and then suspended in an α-MEM at the concentration of 1×10⁴ cells/mL.

The cell suspension thus obtained was placed in wells of a 24-well plate (500 μL each) and pre-incubated at 37° C. for 24 hours. A₁₆S solutions of various concentrations (0-10 μM) were then added to the wells (500 μL each). The wells were irradiated with fluorescence light (27 watts) for 0-60 minutes. The distance between the plate cover and the light source was 5-6 cm. After irradiation, the cells were further incubated for 48 hours. A solution of 3-(4,5-dimethylthiazol-2-yl)-2,5-diphenyl-tetrazolium bromide (0.5% in PBS; 100 μL each) was then added to each cell suspension, thereby causing the cells to produce formazon. After incubation at 37° C. for 3 hours, the suspension medium was discarded. Formazon formed in the cells from each well was extracted with DMSO (1.0 mL), and the DMSO extract solution was immediately measured for absorbance at 540 μm. The absorbance directly correlated with the quantity of formazon, and thus with the quantity of dehydrogenase enzyme or the relative number of the living cells (i.e., viability). The results showed that the cell viability decreased as the A₁₆S dosage and irradiation time increased. With an irradiation time of 60 minutes, a sharp loss of viable cells was observed when A₁₆S concentration was increased from 0 to 2.5 μM. A maximal photodynamic cytotoxicity efficacy of >90% was obtained at an A₁₆S concentration of 5.0-10.0 μM and an irradiation time of 60 minutes. In the absence of light irradiation, no cytotoxicity was observed even at the highest A₁₆S concentration, i.e., 10.0 μM. With the irradiation time of 60 minutes, an unexpectedly low IC₅₀ value of 1.0 μM was observed for A₁₆S.

### EXAMPLE 4

In vivo Photodynamic Therapy Study of A₁₆S.

A photodynamic therapy study was conducted in male ICR mice (Charles River Japan origin Crl: CD-1®(ICR)BR). The mice were 8 weeks old, weighed 37±0.8 g, housed in polycarbonated shoe-box cages on hardwood bedding (5 mice/cage) under controlled pathogen-free conditions (temperature 22±1° C., relative humidity 55±15%, and light/dark cycle 12/12 hours), and allowed free access to a laboratory rodent diet (#5K55, Purina Mills, Inc., St. Louis, Mo.) and water.

Murine sarcoma 180 cells were maintained in the abdominal cavities of other mice with biweekly transplantation. Subcutaneous tumors were induced by intraperitoneal injection of 1×10⁷ tumor cells (about 0.1-0.15 mL ascitic fluid) to the subcutaneous region of the abdominal cavity of each mouse used in the study. The tumor cells were allowed to proliferate at the inoculation site for 5-7 days until the tumors reached a size with a diameter of 10±2 mm. Thirty tumor-bearing mice were divided into 2 groups, i.e., (1) a group treated with A₁₆S (10 mg/kg body weight of each mouse) followed by laser irradiation at 633 nm, and (2) a tumor control group (without treatment with A₁₆S).

Each mouse in group (1) was intraperitoneally injected with A₁₆S in PBS at a site about 2.0 cm away from the tumor location.

The mice in groups (1) and (2) were subsequently kept in the dark for 24 hours, and then anesthetized with avertine (0.3 mL/head). The tumor sites were exposed by removing the hair on and around them and subsequently irradiated with an argon ion laser beam (Spectra Physics, Model 168) at a wavelength of 633 nm for 0-60 minutes. The beam was delivered via a quartz fiber with the circular area of illumination output focused to a diameter of 7-8 mm, with the total light dose adjusted to a level of 100 J/cm² in each experiment. After the irradiation, the body weights and tumor volumes of the mice were measured every 5 days for 30 days. At day 30, all mice were euthanatized by carbon dioxide asphyxiation, and the weight of the tumors were measured.

The results show that tumor cells in the mice in group (2) continuously proliferated at a high rate as expected. The tumor growth rates in the mice in group (1) were much lower than those in group (2) during the entire study period. At day 30, the average tumor weight of the mice in group (1) was about 40% that of the mice in group (2).

### Other Embodiments

A number of embodiments of the invention have been described. Nevertheless, it will be understood that various modifications may be made without departing from the spirit and scope of the invention. For example, the oligoanilines described herein can also be used as photo-activated biocides against fungus and microorganisms. Accordingly, other embodiments are within the scope of the following claims.

What is claimed is:

1. A method of inhibiting the growth of tumor cells in a tumor site of a subject, comprising administering to the tumor site an effective amount of an oligoaniline having the following formula: 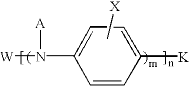
wherein
m is an integer of 1-6;
n is an integer of 1-10;
each A is —H, —Z, —CH₂—CO—OH, —CH₂—CO—O—Z, —CH₂—CO—S—Z, —CH₂—CO—NH₂, or —CH₂—CO—NH—Z; and each X is —H, —O—Z, —S—Z, —NH—Z; Z being —E—D, wherein E is —R—, —R—Ar—, —Ar—R—, or —Ar—; and D is —OH, —SH, —NH₂, —NHOH, —SO₃H, —OSO₃H, —CO₂H, —CONH₂, —CH(NH₂)—CO₂H, —P(OH)₃, —PO(OH)₂, —O—PO(OH)₂, —O—PO(OH)—O—PO(OH)₂, —O—PO(O⁻)—O—CH₂CH₂NH₃⁺, -glycoside, —OCH₃, —OCH₂(CHOH)₄—CH₂OH, —OCH₂(CHOH)₂—CH₂OH, —C₆H₃(OH)₂, —NH₃⁺, —N⁺H₂R_{b}, —N⁺HR_{b}R_{c}, or —N⁺R_{b}R_{c}R_{d}, each of R, R_{b}, R_{c}, and R_{d}, independently, being C₁₋₃₀ alkyl; and Ar being aryl;
W is —H, —CO—B, —CH₂CH(OH)—B, —CO—NH—B, —CS—NH—B, —CO—O—B, CO—CH₂—CH(CO₂H)—B, —CH₂—B, —SO₂—B, wherein B is —R₁—O—[Si(CH₃)₂—O—])₁₋₁₀₀, C₁₋₂₀₀₀ alkyl, C₆₋₄₀ aryl, C₇₋₆₀ alkylaryl, C₇₋₆₀ arylalkyl, (C₁₋₃₀ alkyl ether)₁₋₁₀₀, (C₆₋₄₀ aryl ether)₁₋₁₀₀, (C₇₋₆₀ alkylaryl ether)₁₋₁₀₀, (C₇₋₆₀ arylalkyl ether)₁₋₁₀₀, (C₁₋₃₀ alkyl thioether)₁₋₁₀₀, (C₆₋₄₀ aryl thioether)₁₋₁₀₀, (C₇₋₆₀ alkylaryl thioether)₁₋₁₀₀, (C₇₋₆₀ arylalkyl thioether) ₁₋₁₀₀, (C₂₋₅₀ alkyl ester)₁₋₁₀₀, (C₇₋₆₀ aryl ester)₁₋₁₀₀, (C₈₋₇₀ alkylaryl ester)₁₋₁₀₀, (C₈₋₇₀ arylalkyl ester) ₁₋₁₀₀, —R₁—CO—O—(C₁₋₃₀ alkyl ether) ₁₋₁₀₀, —R₁—CO—O—(C₆₋₄₀ aryl ether) ₁₋₁₀₀, —R₁—CO—O—(C₇₋₆₀ alkylaryl ether)₁₋₁₀₀, —R₁—CO—O—(C₇₋₆₀ arylalkyl ether)₁₋₁₀₀, (C₄₋₅₀ alkyl urethane) ₁₋₁₀₀, (C₁₄₋₆₀ aryl urethane)₁₋₁₀₀, (C₁₀₋₈₀ alkylaryl urethane)₁₋₁₀₀, (C₁₀₋₈₀ arylalkyl urethane)₁₋₁₀₀, (C₅₋₅₀ alkyl urea)₁₋₁₀₀, (C₁₄₋₆₀ aryl urea)₁₋₁₀₀, (C₁₀₋₈₀ alkylaryl urea)₁₋₁₀₀, (C₁₀₋₈₀ arylalkyl urea)₁₋₁₀₀, (C₂₋₅₀ alkyl amide)₁₋₁₀₀, (C₇₋₆₀ aryl amide)₁₋₁₀₀, (C₈₋₇₀ alkylaryl amide)₁₋₁₀₀, (C₈₋₇₀ arylalkyl amide)₁₋₁₀₀, (C₃₋₃₀ alkyl anhydride)₁₋₁₀₀, (C₈₋₅₀ aryl anhydride)₁₋₁₀₀, (C₉₋₆₀ alkylaryl anhydride)₁₋₁₀₀, (C₉₋₆₀ arylalkyl anhydride)₁₋₁₀₀, (C₂₋₃₀ alkyl carbonate)₁₋₁₀₀, (C₇₋₅₀ aryl carbonate)₁₋₁₀₀, (C₈₋₆₀ alkylaryl carbonate)₁₋₁₀₀, (C₈₋₆₀ arylalkyl carbonate)₁₋₁₀₀, —R₁—O—CO—NH—(R₂ or Ar—R₂—Ar)—NH—CO—O—(C₁₋₃₀ alkyl ether, C₆₋₄₀ aryl ether, C₇₋₆₀ alkylaryl ether, or C₇₋₆₀ arylalkyl ether)₁₋₁₀₀, —R₁—O—CO—NH—(R₂ or Ar—R₂—Ar)—NH—CO—O—(C₂₋₅₀ alkyl ester, C₇₋₆₀ aryl ester, C₈₋₇₀ alkylaryl ester, or C₈₋₇₀ arylalkyl ester)₁₋₁₀₀, —R₁—O—CO—NH—(R₂ or Ar—R₂—Ar)—NH—CO—O—(C₁₋₃₀ alkyl ether, C₆₋₄₀ aryl ether, C₇₋₆₀ alkylaryl ether, or C₇₋₆₀ arylalkyl ether)₁₋₁₀₀—CO—NH—(R₂ or Ar—R₂—Ar)—NH—CO—O—, —R₁—O—CO—NH—(R₂ or Ar—R₂—Ar)—NH—CO—O—(C₂₋₅₀ alkyl ester, C₇₋₆₀ aryl ester, C₈₋₇₀ alkylaryl ester, or C₈₋₇₀ arylalkyl ester)₁₋₁₀₀—R₃—O—CO—NH—(R₂ or Ar—R₂—Ar)—NH—CO—O—, —R₁—NH—CO—NH—(R₂ or Ar—R₂—Ar)—NH—CO—O—(C₁₋₃₀ alkyl ether, C₆₋₄₀ aryl ether, C₇₋₆₀ alkylaryl ether, or C₇₋₆₀ arylalkyl ether)₁₋₁₀₀, —R₁—NH—CO—NH—(R₂ or Ar—R₂—Ar)—NH—CO—(C₂₋₅₀ alkyl ester, C₇₋₆₀ aryl ester, C₈₋₇₀ alkylaryl ester, or C₈₋₇₀ arylalkyl ester)₁₋₁₀₀, —R₁—NH—CO—NH—(R₂ or Ar—R₂—Ar)—NH—CO—O—(C1-30 alkyl ether, C₆₋₄₀ aryl ether, C₇₋₆₀ alkylaryl ether, or C₇₋₆₀ arylalkyl ether)₁₋₁₀₀—CO—NH—(R₂ or Ar—R₂—Ar)—NH—CO—O—, —R₁—NH—CO—NH—(R₂ or Ar—R₂—Ar)—NH—CO—O—(C₂₋₅₀ alkyl ester, C₇₋₆₀ aryl ester, C₈₋₇₀ alkylaryl ester, or C₈₋₇₀ arylalkyl ester)₁₋₁₀₀—R₃O—CO—NH—(R₂ or Ar—R₂—Ar)—NH—CO—O—, —R₁—O—CO—NH—(R₂ or Ar—R₂—Ar)—NH—CO—NH—(C₂₋₅₀ alkyl amide, C₇₋₆₀ aryl amide, C₈₋₇₀ alkylaryl amide, or C₈₋₇₀ arylalkyl amide)₁₋₁₀₀, or —R₁—NH—CO—NH—(R₂ or Ar—R₂—Ar)—NH—CO—NH—(C₂₋₅₀ alkyl amide, C₇₋₆₀ aryl amide, C₈₋₇₀ alkylaryl amide, or C₈₋₇₀ arylalkyl amide)₁₋₁₀₀; wherein each of R₁, R₂, and R₃, independently, is C₁₋₃₀ alkyl; and Ar is aryl;
K is —H, —[N(X)—C₆H₄]₁₋₃—NH₂, —[N(X)—C₆H₄]₁₋₃—NH—C(═S)—SH, —[N(X)C₆H₄]₁₋₃—N═CH—Ar—SH, or —[N(X)—C₆H₄]₁₋₃—NH—CO—Ar—SH, wherein X is —H, —Z, —CH₂—CO—OH, —CH₂—CO—O—Z, —CH₂—CO—S—Z, —CH₂—CO—NH₂ or —CH₂—CO—NH—Z; and Ar is aryl;
and subsequently exposing the tumor site to irradiation.

2. The method of claim 1, wherein A is —Z, —CH₂—CO—O—Z, —CH₂—CO—S—Z, or —CH₂—CO—NH—Z; wherein E is —R— or —R—Ar—; and D is —OH, —SH, —NH₂, —NHOH, —SO₃H, —OSO₃H, —CO₂H, —CONH₂, —CH(NH₂)—CO₂H, —P(OH)₃, —PO(OH)₂, —O—PO(OH)₂, —O—PO(OH)—O—PO(OH)₂, or —NH₃⁺.

3. The method of claim 1, wherein m is an integer of 2-6.

4. The method of claim 1, wherein n is an integer of 1-6.

5. The method of claim 2, wherein A is —Z, Z being —E—D, wherein E is —R—, or —R—Ar—; and D is —OH, —SH, —NH₂, —NHOH, —SO₃H, —OSO₃H, —CO₂H, —CONH₂, —P(OH)₃, —PO(OH)₂, —O—PO(OH)₂, —O—PO(OH)—O—PO(OH)2, or —NH₃⁺.

6. The method of claim 2, wherein n is an integer of 1-6.

7. The method of claim 2, wherein m is an integer of 2-6.

8. The method of claim 6, wherein m is an integer of 2-6.

9. The method of claim 5, wherein E is —R—; and D is —SO₃H, —OSO₃H, —CO₂H, —O—PO(OH)₂, or —O—PO(OH)—O—PO(OH)₂.

10. The method of claim 5, wherein m is an integer of 2-6.

11. The method of claim 5, wherein n is an integer of 1-6.

12. The method of claim 9, wherein E is —C₃H₆—; D is —SO₃H; n is an integer of 1-6; and m is an integer of 2-6.

13. The method of claim 12, wherein m is 4.

14. The method of claim 13, wherein each of W, X, and K is H.

15. The method of claim 3, wherein m is 4.

16. The method of claim 3, wherein n is an integer of 1-6.

17. The method of claim 15, wherein n is an integer of 1-6.

18. A pharmaceutical composition for inhibiting the growth of tumor cells, comprising a compound of the following formula: 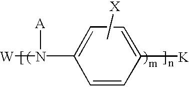
wherein
m is an integer of 1-6;
n is an integer of 1-10;
each A is —H, —Z, —CH₂—CO—OH, —CH₂—CO—O—Z, —CH₂—CO—S—Z, —CH₂—CO—NH₂, or —CH₂—CO—NH—Z; and each X is —H, —O—Z, —S—Z, —NH—Z; Z being —E—D, wherein E is —R—, —R—Ar—, —Ar—R—, or —Ar—; and D is —OH, —SH, —NH₂, —NHOH, —SO₃H, —OSO₃H, —CO₂H, —CONH₂, —CH(NH₂)—CO₂H, —P(OH)₃, —PO(OH)₂, —O—PO(OH)₂, —O—PO(OH)—O—PO(OH)₂, —O—PO(O⁻)—O—CH₂CH₂NH₃⁺, -glycoside, —OCH₃, —OCH₂(CHOH)₄—CH₂OH, —OCH₂(CHOH)₂—CH₂OH, —C₆H₃(OH)₂, —NH₃⁺, —N⁺H₂R_{b}, —N⁺HR_{b}R_{c}, or —N⁺R_{b}R_{c}R_{d}, each of R, R_{b}, R_{c}, and R_{d}, independently, being C₁₋₃₀ alkyl; and Ar being aryl;
W is —H, —CO—B, —CH₂CH(OH)—B, —CO—NH—B, —CS—NH—B, —CO—O—B, CO—CH₂—CH(CO₂H)—B, —CH₂—B, —SO₂—B, wherein B is —R₁—O—[Si(CH₃)₂—O—])₁₋₁₀₀, C₁₋₂₀₀₀ alkyl, C₆₋₄₀ aryl, C₇₋₆₀ alkylaryl, C₇₋₆₀ arylalkyl, (C₁₋₃₀ alkyl ether)₁₋₁₀₀, (C₆₋₄₀ aryl ether)₁₋₁₀₀, (C₇₋₆₀ alkylaryl ether)₁₋₁₀₀, (C₇₋₆₀ arylalkyl ether)₁₋₁₀₀, (C₁₋₃₀ alkyl thioether)₁₋₁₀₀, (C₆₋₄₀ aryl thioether)₁₋₁₀₀, (C₇₋₆₀ alkylaryl thioether)₁₋₁₀₀, (C₇₋₆₀ arylalkyl thioether)₁₋₁₀₀, (C₂₋₅₀ alkyl ester)₁₋₁₀₀, (C₇₋₆₀ aryl ester)₁₋₁₀₀, (C₈₋₇₀ alkylaryl ester)₁₋₁₀₀, (C₈₋₇₀ arylalkyl ester)₁₋₁₀₀, —R₁—CO—O—(C₁₋₃₀ alkyl ether)₁₋₁₀₀, —R₁—CO—O—(C₆₋₄₀ aryl ether)₁₋₁₀₀, —R₁—CO—O—(C₇₋₆₀ alkylaryl ether)₁₋₁₀₀, —R₁—CO—O—(C₇₋₆₀ arylalkyl ether)₁₋₁₀₀, (C₄₋₅₀ alkyl urethane)₁₋₁₀₀, (C₁₄₋₆₀ aryl urethane)₁₋₁₀₀, (C₁₀₋₈₀ alkylaryl urethane)₁₋₁₀₀, (C₁₀₋₈₀ arylalkyl urethane)₁₋₁₀₀, (C₅₋₅₀ alkyl urea)₁₋₁₀₀, (C₁₄₋₆₀ aryl urea)₁₋₁₀₀, (C₁₀₋₈₀ alkylaryl urea)₁₋₁₀₀, (C₁₀₋₈₀ arylalkyl urea)₁₋₁₀₀, (C₂₋₅₀ alkyl amide)₁₋₁₀₀, (C₇₋₆₀ aryl amide)₁₋₁₀₀, (C₈₋₇₀ alkylaryl amide)₁₋₁₀₀, (C₈₋₇₀ arylalkyl amide)₁₋₁₀₀, (C₃₋₃₀ alkyl anhydride)₁₋₁₀₀, (C₈₋₅₀ aryl anhydride)₁₋₁₀₀, (C₉₋₆₀ alkylaryl anhydride)₁₋₁₀₀, (C₉₋₆₀ arylalkyl anhydride)₁₋₁₀₀, (C₂₋₃₀ alkyl carbonate)₁₋₁₀₀, (C₇₋₅₀ aryl carbonate)₁₋₁₀₀, (C₈₋₆₀ alkylaryl carbonate)₁₋₁₀₀, (C₈₋₆₀ arylalkyl carbonate)₁₋₁₀₀, —R₁—O—CO—NH—(R₂ or Ar—R₂—Ar)—NH—CO—O—(C₁₋₃₀ alkyl ether, C₆₋₄₀ aryl ether, C₇₋₆₀ alkylaryl ether, or C₇₋₆₀ arylalkyl ether)₁₋₁₀₀, —R₁—O—CO—NH—(R₂ or Ar—R₂—Ar)—NH—CO—O—(C₂₋₅₀ alkyl ester, C₇₋₆₀ aryl ester, C₈₋₇₀ alkylaryl ester, or C₈₋₇₀ arylalkyl ester)₁₋₁₀₀, —R₁—O—CO—NH—(R₂ or Ar—R₂—Ar)—NH—CO—O—(C₁₋₃₀ alkyl ether, C₆₋₄₀ aryl ether, C₇₋₆₀ alkylaryl ether, or C₇₋₆₀ arylalkyl ether)lloo—CO—NH—(R₂ or Ar—R₂—Ar)—NH—CO—O—, —R₁—O—CO—NH—(R₂ or Ar—R₂—Ar)—NH—CO—O—(C₂so alkyl ester, C₇₋₆₀ aryl ester, C₈₋₇₀ alkylaryl ester, or C₈₋₇₀ arylalkyl ester)₁₋₁₀₀—R₃—O—CO—NH—(R₂ or Ar—R₂—Ar)—NH—CO—O—, —R₁—NH—CO—NH—(R₂ or Ar—R₂—Ar)—NH—CO—O—(C₁₋₃₀ alkyl ether, C₆₋₄₀ aryl ether, C₇₋₆₀ alkylaryl ether, or C₇₋₆₀ arylalkyl ether)₁₋₁₀₀, —R₁—NH—CO—NH—(R₂ or Ar—R₂—Ar)—NH—CO—(C₂₋₅₀ alkyl ester, C₇₋₆₀ aryl ester, C₈₋₇₀ alkylaryl ester, or C₈₋₇₀ arylalkyl ester)₁₋₁₀₀, —R₁—NH—CO—NH—(R₂ or Ar—R₂—Ar)—NH—CO—O—(C₁₋₃₀ alkyl ether, C₆₋₄₀ aryl ether, C₇₋₆₀ alkylaryl ether, or C₇₋₆₀ arylalkyl ether)₁₋₁₀₀—CO—NH—(R₂ or Ar—R₂—Ar)—NH—CO—O—, —R₁—NH—CO—NH—(R₂ or Ar—R₂—Ar)—NH—CO—O—(C₂₋₅₀ alkyl ester, C₇₋₆₀ aryl ester, C₈₋₇₀ alkylaryl ester, or C₈₋₇₀ arylalkyl ester)₁₋₁₀₀—R₃O —CO—NH—(R₂ or Ar—R₂—Ar)—NH—CO—O—, —R₁—O—CO—NH—(R₂ or Ar—R₂—Ar)—NH—CO—NH—(C₂₋₅₀ alkyl amide, C₇₋₆₀ aryl amide, C₈₋₇₀ alkylaryl amide, or C₈₋₇₀ arylalkyl amide)₁₋₁₀₀, or —R₁—NH—CO—NH—(R₂ or Ar—R₂—Ar)—NH—CO—NH—(C₂₋₅₀ alkyl amide, C₇₋₆₀ aryl amide, C₈₋₇₀ alkylaryl amide, or C₈₋₇₀ arylalkyl amide)₁₋₁₀₀; wherein each of R₁, R₂, and R₃, independently, is C₁₋₃₀ alkyl; and Ar is aryl;
K is —H, —[N(X)—C₆H₄]₁₋₃—NH₂, —[N(X)—C₆H₄]₁₋₃—NH—C(═S)—SH, —[N(X)—C₆H₄]₁₋₃—N═CH—Ar—SH, or —[N(X)—C₆H₄]₁₋₃—NH—CO—Ar—SH, wherein X is —H, —Z, —CH₂—CO—OH, —CH₂—CO—O—Z, —CH₂—CO—S—Z, —CH₂—CO—NH₂ or —CH₂—CO—NH—Z; and Ar is aryl; and
a pharmaceutically acceptable carrier

19. The pharmaceutical composition of claim 18, wherein A is —Z. —CH₂—CO—O—Z, —CH₂—CO—S—Z, or —CH₂—CO—NH—Z; E is —R— or —R—Ar—; and D is —OH, —SH, —NH₂, —NHOH, —SO₃H, —OSO₃H, —CO₂H, —CONH₂, —CH(NH₂)—CO₂H, —P(OH)₃, —PO(OH)₂, —O—PO(OH)₂, —O—PO(OH)—O—PO(OH)₂, or —NH₃⁺.

20. The pharmaceutical composition of claim 19, wherein A is —Z; E is —R—; and D is —SO₃H, —OSO₃H, —CO₂H, —CH(NH₂)—CO₂H, —P(OH)₃, —PO(OH)₂, —O—PO(OH)₂, or —O—PO(OH)—O—PO(OH)₂.

21. The pharmaceutical composition of claim 20, wherein E is —C₃H₆—; D is —SO₃H; n is an integer of 1-6; m is an integer of 2-6; and each of W, X, and K is H.
